(19) United States
(12) Patent Application Publication
(10) Pub. No.: US 2016/0015745 A1
(43) Pub. Date: Jan. 21, 2016

(54) Formulations of Antimicrobial, Antiviral, and Antineoplastic Compounds In Combination With Certain Wavelengths of Light

(71) Applicant: Paul Dabney, Georgetown, TX (US)

(72) Inventor: Paul Dabney, Georgetown, TX (US)

(21) Appl. No.: 14/697,579

(22) Filed: Apr. 27, 2015

Related U.S. Application Data
(63) Continuation-in-part of application No. 14/497,269, filed on Sep. 25, 2014.
Application No. 14/497,269 is a continuation-in-part of application No. 14/536,633, filed on Nov. 9, 2014.
Application No. 14/536,633 is a continuation-in-part of application No. 14/583,580, filed on Dec. 26, 2014.
Application No. 14/583,580 is a continuation-in-part of application No. 14/630,513, filed on Feb. 24, 2015.
Application No. 14/630,513 is a continuation-in-part of application No. 14/638,902, filed on Mar. 4, 2015.
(60) Provisional application No. 62/026,498, filed on Jul. 18, 2014.

(51) Int. Cl. A61K 33/40; A61K 38/48; A61K 38/51; A61K 31/155; A61K 41/00; A61K 33/00; A61K 45/06; A61K 31/496; A61K 31/02; A61N 5/06; A61K 38/19

(52) U.S. Cl. CPC A61K 33/40; A61N 5/062; A61N 5/0624; A61K 38/48; A61K 38/51; A61K 38/486; A61K 31/155; A61K 38/191; A61K 33/00; A61K 45/06; A61K 31/496; A61K 31/02; A61K 41/00; A61N 2005/0651; A61N 2005/067

(57) ABSTRACT

Formulations of antimicrobial, antiviral, and antineoplastic compounds in combination with certain wavelengths of light include a solution that includes an oxidizer; the solution having an initial therapeutic effectiveness; and a light of a predetermined frequency that that undergoes a synergistic reaction with the oxidizer, thereby enhancing the initial therapeutic effectiveness of the solution. A method of drug-enhancement includes providing a drug that includes hydrogen peroxide and a therapeutic chemical, the drug having an initial therapeutic effectiveness; and applying light having a predetermined frequency of light that has a synergistic reaction with the drug, thereby enhancing the initial therapeutic effectiveness of the drug.

### RELATED APPLICATIONS

This application claims the benefit of the filing date of U.S. Patent Application No. 62/026,498, filed Jul. 18, 2014, (P679-104) which is incorporated herein by reference in its entirety, U.S. patent application Ser. No. 14/497,269, filed Sep. 25, 2014, (P679-104A) which is incorporated herein by reference in its entirety; U.S. patent application Ser. No. 14/536,633, filed Nov. 9, 2014, (P679-104B) which is incorporated herein by reference in its entirety; U.S. patent application Ser. No. 14/583,580, filed Dec. 26, 2014, (P679-104C) which is incorporated herein by reference in its entirety; U.S. patent application Ser. No. 14/630,513, filed Feb. 24, 2015, (P679-104E) which is incorporated herein by reference in its entirety; and U.S. patent application Ser. No. 14/638,902, filed Mar. 4, 2015, (P679-104F) which is incorporated herein by reference in its entirety.

### BACKGROUND OF THE INVENTION

The present invention generally relates to formulations of antimicrobial and/or antiviral and/or antineoplastic compounds in combination with certain wavelengths of light to form a synergistic reaction to treat a condition or conditions.

Light of certain wavelengths has been proven to kill, slow and/or stop microbial infections. Light of certain wavelengths has been proven to kill and/or stop cancer growth. Light of certain wavelengths had been proven to kill and/or stop viral infections. Light is an ingredient that kills and/or stops microbial and/or viral and/or cancer growth.

In general, patient compliance is a problem with medications and/or treatments that require a dosing regimen. There is desire for enhancing treatment regimens and further improving patient results. The development of a novel antibacterial and/or antiviral and/or antineoplastic formulation that includes a synergistic reaction using a certain wavelength of light that amplifies the effect of certain chemicals would be an advance in the art. Increasing the effectiveness of a drug by the addition of a certain wavelength of light is a means of modifying its biological properties. As yet, there is no antimicrobial and/or antiviral and/or antineoplastic treatment that includes a synergistic reaction between light of a certain wavelength and a chemical or chemicals.

Microbes exist that cause harm or disease in living tissues of animals.

Light of certain wavelengths has been demonstrated to improve or “super-charge” the effects of certain chemicals, including antimicrobial agents and other target chemicals, creating a synergistic effect upon the chemical.

Most chemical reactions work best at a certain temperature. These ideal temperatures vary for each reaction. A “scalding chart” might indicate that water of 130 degrees is safe under an exposure of 30 seconds, but over that it causes burns. Water of 120 degrees may be safe up to 5 minutes. By optimizing the temperature, the temperature range that best suits the application is utilized.

It would be desirable to add light of certain wavelengths to drugs having a beneficial effect, so a synergistic effect can be created to enhance the beneficial effect.

### SUMMARY OF THE INVENTION

In one aspect of the present invention, a formulation includes a solution that includes an oxidizer; the solution having an initial therapeutic effectiveness; and a light of a predetermined frequency that that undergoes a synergistic reaction with the oxidizer, thereby enhancing the initial therapeutic effectiveness of the solution.

In another aspect of the present invention, a solution includes hydrogen peroxide; a target chemical that includes an antimicrobial, biocidal, disinfectant, antineoplastic, antibiotic, antispasmodic, or non-steroidal anti-inflammatory; and a light of a predetermined frequency that that undergoes a synergistic reaction with the solution, thereby enhancing a therapeutic effectiveness of the solution.

In yet another aspect of the present invention, a method of drug-enhancement includes providing a drug that includes hydrogen peroxide and a therapeutic chemical, the drug having an initial therapeutic effectiveness; and applying light having a predetermined frequency of light that has a synergistic reaction with the drug, thereby enhancing the initial therapeutic effectiveness of the drug.

### DETAILED DESCRIPTION

This application does not include any drawings. The preferred embodiments can be described utilizing words alone.

The preferred embodiment and other embodiments, which can be used in industry and include the best mode now known of carrying out the invention, are hereby described in detail. Further embodiments, features and advantages will become apparent from the ensuing description, or may be learned without undue experimentation. The following description of embodiments, even if phrased in terms of “the invention” or what the embodiment “is,” is not to be taken in a limiting sense, but describes the manner and process of making and using the invention. The coverage of this patent will be described in the claims. The order in which steps are listed in the claims does not necessarily indicate that the steps must be performed in that order.

Embodiments of the present invention generally provide a drug having an effectiveness that is enhanced by shining light of a predetermined frequency onto the drug.

There is a need for pharmaceutically acceptable compounds that treat a broad spectrum of bacterial, viral and/or antineoplastic conditions. Accordingly, the present invention relates to forms comprising an effective amount of a pharmaceutically acceptable antimicrobial and/or antiviral and/or antineoplastic compounds in a synergistic reaction with certain wavelengths of light. The present invention also relates to the use of such dosage forms for the treatment of one or more conditions in a subject suitable for treatment by antimicrobial and/or antiviral and/or antineoplastic pharmaceutically acceptable ingredients in conjunction with light of certain wavelengths.

Hydrogen peroxide (H2O2) can be either an oxidizing agent or a reducing agent. When H2O2 serves as an oxidizing agent, the oxygen is reduced to H2O. When H2O2 serves as a reducing agent, the oxygen is oxidized to O2 and bubbles are noticed. Embodiments of the present invention may utilize H2O2 and formulations of H2O2 as an oxidizing agent or a reducing agent to provide an antimicrobial effect, antineoplastic effect and/or antiviral effect. A formulation or drug will include an “oxidizer” if it includes H2O2.

An embodiment of the drug consists of a number of ingredients that include light of certain wavelengths and chemicals that alone or in combinations inhibit, retard and/or stop microbial, and/or viral and/or neoplastic growth. The drug may resemble antimicrobial and/or antiviral and/or antineoplastic chemicals in terms of its chemical and functionality profiles in vivo. The drug may have light as one of its ingredients. It reliably produces antimicrobial and/or antiviral and/or antineoplastic like effects in humans and other animals at clinically prescribed doses. It is designated as light of a certain wavelength containing at least one or more of the following chemicals; H2O2, lauric acid, dodecanoic acid, topical antibiotics, topical anesthetics, nicotinic acid, nicotinamide, antimicrobials such as clindamycin phosphate, Methyl 7-chloro-6,7,8-trideoxy-6-(1-methyl-trans-4-propyl-L-2-pyrrolidinecarboxamido) -1-thio-L-threo-α-D-galacto-octopyranoside 2-(dihydrogen phosphate), salicylic acid, sulfur, retinoids such as 6-[3-(1-adamantyl)-4-methoxy-phenyl] naphthalene-2-carboxylic acid, Alpha Hydroxy acids, tretinoin, borax, caprylic acid, capric acid, myristic acid and additional chemicals useful in said method.

The present invention relates to forms of an antimicrobial and/or antiviral and/or antineoplastic drug comprising an effective amount of a pharmaceutically acceptable chemical or chemicals that are more effective than otherwise when exposed to certain wavelengths of light due to a synergistic reaction. This synergistic reaction enables treatment to occur with a lower concentration of chemicals or a greater effect with the same concentration of chemicals. This increased effectiveness is novel to this invention.

The dosage forms of the present invention may comprise a compound consisting of certain wavelengths of light and one or more of the following chemicals: H2O2, lauric acid, dodecanoic acid, topical antibiotics, topical anesthetics, nicotinic acid, nicotinamide, antimicrobials such as clindamycin phosphate, Methyl 7-chloro-6,7,8-trideoxy-6-(1-methyl-trans-4-propyl-L-2-pyrrolidinecarboxamido) -1-thio-L-threo-α-D-galacto-octopyranoside 2-(dihydrogen phosphate), salicylic acid, sulfur, retinoids such as 6-[3-(1-adamantyl)-4-methoxy-phenyl] naphthalene-2-carboxylic acid, Alpha Hydroxy acids, tretinoin, borax, caprylic acid, capric acid, myristic acid and additional chemicals useful in said method including pharmaceutically acceptable carriers.

Embodiments of a drug that treats microbial infections, and/or neoplasms and/or viral infections by combining light of a certain wavelength with a chemical or chemicals may be a new way of treating infections and/or neoplasms.

Embodiments may enhance the effect of a solution containing a combination of antimicrobials, antineoplastics, antivirals, or other pharmaceutical target chemicals. The light is like an ingredient, in that it may be used to supercharge the target chemical before or after the solution is applied for therapy.

In an embodiment, therapy may be applied to humans, animals, or other targets that benefit from the supercharged target chemical. The light may be applied to a therapeutic chemical or solution that has already been placed in contact with human or animal tissue, or the light may be applied to the target chemical before the chemical is used for therapy. A “chemical” may include any chemical element or solution of chemicals.

In an embodiment, the therapy may include multiple frequencies of light, which may be applied in a series or at the same time. An example embodiment may include a first frequency that is known to enhance an anti-microbial such as hydrogen peroxide, and a second frequency that is known to enhance another pharmaceutical chemical such as an anti-neoplastic or another anti-microbial. Embodiments of the invention are not limited to direct application to tissue, and may include enhancement of a target chemical's pharmaceutical or biological properties with predetermined frequencies of light that enhance or supercharge the target chemical.

In an embodiment, a solution may be chosen that includes a therapeutic or target chemical in combination with an oxidizer or other anti-microbial. The target solution may then be exposed to one or more predetermined frequencies of light that have a synergistic reaction with the solution, thereby enhancing the effectiveness of the solution.

Once applied, the solution including hydrogen peroxide or other antimicrobial chemical is exposed to a wavelength of light that creates a synergistic effect enhancing the effectiveness of the antimicrobial chemical. This synergistic effect causes a greater reduction in bacteria associated with bacteria than the applications of the antimicrobial solution alone or the light alone.

An embodiment of the present invention generally provides a device to hold solutions in contact with tissues, such as an animal's flesh, while the tissues and solutions are simultaneously being exposed to certain wavelengths of light. This device has a component that amplifies the effect of the antimicrobial solutions by using a certain wavelength of light. The antimicrobial solution may or may not be light activated at any given time. When the light is on, the solution is “supercharged” by the light. This synergistic effect eliminates or reduces more microbes than the solution acting alone.

Microbes exist that cause harm or disease in living tissues. By adding a light of certain wavelengths to a device that holds certain antimicrobial agents in close proximity to tissues, a synergistic effect can be created to destroy or inhibit microbial growth. In the oral cavity, this device could be a tray designed to cover the teeth and gingival. This tray would emit certain wavelengths of light that when combined with certain antimicrobial solutions in the tray would cause a synergistic antimicrobial effect. The light could be produced, for example, from a light emitting diode (LED) or laser. An external light source could be connected to the fiber optic cable in the solution holding apparatus with a fiber optic connection cable that may also include a fiber optic connection interface or plug.

Embodiments of the present invention may create another means to treat disease. Super charging antimicrobial solutions with certain wavelengths of lights may cause the solutions to eliminate or reduce microbes at a higher percentage than the solution alone.

Embodiments may create a synergistic effect between certain wavelengths of light and antimicrobial solutions that when applied to tissues eliminates or reduces disease causing microorganisms.

Embodiments of the present invention may consist of a solution holding apparatus or medium that emits certain wavelengths of light into the solution. When this light and solution combination is applied to tissues, a synergistic effect is created that reduces or eliminates microorganisms that cause disease. The essential components are 1. The solution holding apparatus 2. A light source 3. An antimicrobial solution.

Embodiments may utilize blue light, or another certain predetermined wavelength of light that supercharges the solution, with an exposure from a few second to minutes. Embodiments may also use an H2O2 solution, such as a gel, with concentration of 0.3 mM or any concentration of solution that is suitable as an antimicrobial agent.

In an embodiment, for safety, a “scalding chart” might indicate that water of 130 degrees is safe under an exposure of 30 seconds, but over that it causes burns. Water of 120 degrees may be safe up to 5 minutes. Hydrogen peroxide (H2O2), when it is exposed to a light of 400-500 nanometers wavelength, may kill 96% of microbes in less than 20 seconds. This solution may work best at 57 degrees Celsius (134 degrees F.).

Alternate embodiments may include heating elements that warm and further super-charge the antimicrobial solution. In embodiments, a device may contain heating or cooling components or both. In an embodiment, an antimicrobial solution may be preheated to an ideal or optimal temperature before it is exposed to synergizing light. For example, Hydrogen peroxide may preferably be exposed to a light of 400-500 nanometers at 57 degrees Celsius (134 degrees F.) for less than 20 seconds. Other chemicals may have different preferred temperatures.

Embodiments of a treatment may include drugs that have a synergistic reaction with hydrogen peroxide. A first drug may include an antimicrobial. A second drug may include an anticancer drug. Other drugs may include medicine or pharmaceuticals that provide a beneficial effect. Embodiments of drugs may be combined with light, to produce a synergistic effect. Light is an ingredient in the treatment. The light may be supplied by a light source, or by a device that applies the drug and the light.

Embodiments may include light undergoing a synergistic reaction with an oxidizer, such as, for example, hydrogen peroxide. The embodiment may also include another drug or target chemical that undergoes a synergistic reaction with the light, thereby providing a treatment that includes light, an oxidizer, and the target chemical, where the light and oxidizer has enhances the effect of the target chemical. Embodiments of a medical drug may include an antimicrobial, an anticancer drug, or another pharmaceutical.

Embodiments of a target chemical may include a compound with one or more of an antimicrobial, a biocidal or a disinfectant. Embodiments of an antimicrobial may include a chlorhexidine compound.

Embodiments of a target chemical may include a non-steroidal anti-inflammatory compound, such as, for example, an indene derivative.

Embodiments of a target chemical may include an iodine compound.

Embodiments of a target chemical may include a Tumor Necrosis Factor or a member of the TNF family of cytokines.

Embodiments of a target chemical may include a metal such as silver (Ag) or a transition metal such as Cu or Fe.

Embodiments of a target chemical may include an enzyme. Embodiments of an enzyme may include one or more of protease, pectinase, or elastase.

Embodiments of a target chemical may include an antineoplastic.

Embodiments of a target chemical may include an antibiotic, such as, for example, ciprofloxacin.

Embodiments of a target chemical may include an antispasmodic, such as, for example, methylene bicloride.

In an embodiment, a first reaction may include light of a certain wavelength, such as 400-500 nanometers, combined with an oxidizer, such as hydrogen peroxide. The light plus oxidizer may be a treatment all by itself A second reaction may include light of a certain wavelength combined with an oxidizer, further combined with a target chemical undergoing a synergistic reaction. Embodiments of a drug may include an oxidizer that has been light-enhanced, and a target chemical that has been light-enhanced, to provide a drug that has a light-enhanced therapeutic effectiveness. The drug may be a solution that is held in contact with a user while the light is applied is applied from a light source, or the solution may be light-enhanced before application. The light may include two frequencies of light that are applied to simultaneously applied to a drug or solution, or a first frequency of light may be applied and then a second frequency of light may be applied in series.

The selected target chemical may be used in combination with surfactants, wetting agents chelating agents, or useful ingredients. Embodiments of medical drugs may be used in tablet, pill, capsule, gel, liquid, spray, mist, cream, or paste form. Embodiments may be used at varying temperatures to modulate their efficacy.

Embodiments may include a solution-holding apparatus, a light source that generates the light and applies the light to the solution; and a heating element. An embodiment of the present invention may include a covering for a human, horse or other animal. The covering may be connected to a light source. Embodiments may contain a multitude of fiber optic terminations. Coverings may have a heating element. Embodiments of the present invention may include a dental tray that retains an antimicrobial or antineoplastic solution against a user's teeth; a medical solution retainer adapted to retain an antimicrobial or antineoplastic solution against a human user's tissue; a bucket or container; a bowl; a full body suit; an arm sleeve; a glove; a leg stocking; a toe cap; a helmet; a catheter tube; a medical solution retainer adapted to retain an antimicrobial or antineoplastic solution against an animal's tissue; a blanket for horses or other animals; or a covering for limbs or parts of an animal.

I claim:

1. A formulation comprising:
a solution that includes an oxidizer; the solution having an initial therapeutic effectiveness; and
a light of a predetermined frequency that that undergoes a synergistic reaction with the oxidizer, thereby enhancing the initial therapeutic effectiveness of the solution.

2. The formulation of claim 1, wherein the oxidizer is hydrogen peroxide.

3. The formulation of claim 1, wherein the solution includes a target chemical combined with the oxidizer and the combined solution undergoes a synergistic reaction with the light.

4. The formulation of claim 3, wherein the target chemical is an antimicrobial, a biocidal, or a disinfectant compound.

5. The formulation of claim 3, wherein the target chemical is an antimicrobial compound that includes chlorhexidine.

7. The formulation of claim 3, wherein the target chemical is a non-steroidal anti-inflammatory compound that includes an indene derivative.

8. The formulation of claim 3, wherein the target chemical is an iodine compound.

9. The formulation of claim 3, wherein the target chemical is Tumor Necrosis Factor.

10. The formulation of claim 3, wherein the target chemical is a metal.

11. The formulation of claim 3, wherein the target chemical is a transition metal.

12. the formulation of claim 3, wherein the target chemical is an enzyme.

13. The formulation of claim 3, wherein the target chemical is a protease, pectinase, or elastase compound.

14. The formulation of claim 3, wherein the target chemical is antineoplastic.

15. The formulation of claim 3, wherein the target chemical is an antibiotic.

16. The formulation of claim 3, wherein the target chemical is ciprofloxacin.

17. The formulation of claim 3, wherein the target chemical is an antispasmodic.

18. The formulation of claim 3, wherein the target chemical is methylene bicloride.

19. A solution comprising:
hydrogen peroxide;
a target chemical that includes an antimicrobial, biocidal, disinfectant, antineoplastic, antibiotic, antispasmodic, or non-steroidal anti-inflammatory; and
a light of a predetermined frequency that that undergoes a synergistic reaction with the solution, thereby enhancing a therapeutic effectiveness of the solution.

20. A method of drug-enhancement, comprising:
providing a drug that includes hydrogen peroxide, the drug having an initial therapeutic effectiveness; and
applying a predetermined frequency of light that has a synergistic reaction with the hydrogen peroxide, thereby enhancing the initial therapeutic effectiveness of the drug.
